(19) United States
(12) Patent Application Publication
(10) Pub. No.: US 2024/0206904 A1
(43) Pub. Date: Jun. 27, 2024

(54) Ultrasonic System for Ultrasonic Surgery and Methods for its Operation

(71) Applicant: SÖRING GMBH, Quickborn (DE)

(72) Inventors: Florian NEUMANN, Hamburg (DE); Steffen BUSCHSCHLÜTER, Hemdingen (DE); Gesa MALTZEN, Heilshoop (DE)

(21) Appl. No.: 18/392,616

(22) Filed: Dec. 21, 2023

(30) Foreign Application Priority Data
Dec. 23, 2022 (EP) 22216318.0

(51) Int. Cl. A61B 17/32; A61B 17/00

(52) U.S. Cl. CPC A61B 17/320068; A61B 2017/00137; A61B 2017/00199; A61B 2017/320069; A61B 2017/32007; A61B 2217/007

(57) ABSTRACT

An ultrasonic system for ultrasonic surgery including an ultrasonic instrument having a sonotrode and a piezoelectric converter, an ultrasonic generator driving the converter, and a device communicating with the ultrasonic instrument, for the supply of an irrigation fluid to the ultrasonic instrument at a predetermined irrigation rate and/or for effecting a predetermined negative pressure applied to the ultrasonic instrument. The negative pressure is configured for the aspiration of tissue fragmented by the ultrasonic instrument. A controller sets a current ablation rate between a predetermined minimum ablation rate and a predetermined maximum ablation rate depending on the tissue type. The ablation rates are respectively determined by a value pair including sonotrode amplitude, and irrigation rate or negative pressure, or a value triplet including sonotrode amplitude, irrigation rate, and negative pressure.

### CROSS-REFERENCE TO RELATED APPLICATIONS

This application claims benefit of European Patent Application No. 22 216 318.0 filed on Dec. 23, 2022. The entire content of this application is incorporated herein by reference.

### BACKGROUND OF THE INVENTION

The invention relates to an ultrasonic system configured for ultrasonic surgery and a method for its operation. In particular, the invention relates to an ultrasonic system for ultrasonic surgery with an ultrasonic instrument including a sonotrode and a piezoelectric converter, an ultrasonic generator driving the converter, and a device, communicating with the ultrasonic instrument, for the supply of an irrigation fluid to the ultrasonic instrument at a predetermined irrigation rate and/or for effecting a predetermined negative pressure applied to the ultrasonic instrument, which negative pressure is configured for the aspiration of tissue fragmented by the ultrasonic instrument. In addition, the invention specifically relates to a method for operating such an ultrasonic system by generating a predetermined sonotrode amplitude, supplying an irrigation fluid to the ultrasound instrument at a predetermined irrigation rate, and/or effecting a predetermined negative pressure applied to the ultrasonic instrument, which negative pressure is configured for the aspiration of tissue fragmented by the ultrasonic instrument, for effecting a predetermined ablation rate.

In ultrasonic surgery, especially in liver surgery, neurosurgery, and spinal column surgery, ultrasonic systems are used for the coarse ablation and fine preparation of tissues. The known ultrasonic systems designed for ultrasonic surgery consist in particular of an ultrasonic generator that transmits alternating current at a very high frequency to an ultrasonic instrument provided with a device for irrigation and/or aspiration. By means of piezoelectric ceramics the ultrasonic instrument converts the electrical energy into mechanical movement, which is amplified via a sonotrode. Depending on the technical construction, contact between vibrating sonotrode and the tissue results in a tissue effect in the form of fragmentation, ablation, coagulation, or dissection. The extent of these effects depends on the one hand on the energy input of the sonotrode, and on the other hand on the acoustic and biological properties of the tissue, which are ultimately reflected in their mechanical properties. Soft tissue (e.g., liver parenchyma) requires a lower energy input than strongly elastic (e.g., blood vessels) or hard tissue (e.g., bones).

This dependence of the tissue effect on the tissue type is one of the advantages of using ultrasonic systems in ultrasonic surgery. When using ultrasonic instruments, surgical treatments can also be carried out on the one hand in close proximity to highly sensitive structures. On the other hand, there is no strict correlation between the parameters specified by the operator and the extent of the tissue effect.

A fundamental disadvantage of the known systems is therefore that a successful surgical treatment requires the surgeon to have a sufficient degree of experience in operating the known ultrasonic systems. In the clinical practice, surgeons work with a range of “tissue ablation speeds” that can be understood in the technical sense as ablation or dissection rates. In the proximity of critical structures, such as, for example, blood vessels, the resection is slow in order to minimize the risk of complications. In principle, however, the resection should also be carried out in an acceptable time with an acceptable effort in order to minimize the surgery time and the stress on the patient. On the one hand, there is therefore a lower limit (“minimum ablation rate”) for the desired tissue effect, on the other hand, however, the resection should also be expeditious, although not uncontrolled, so that an upper limit (“maximum ablation rate”) exists for the desired tissue effect at the same time.

The setting of the “tissue ablation speed” or ablation rate sought for by the surgeon is performed by the surgeon based on experience, or intuitively depending on the surgical field discovered in situ by means of an ultrasonic instrument at a predetermined ultrasound frequency (in the range of 20 kHz to 60 kHz) at the ultrasonic generator. As parameters the amplitude of the sonotrode (in % of the maximum amplitude; also referred to as “ultrasound”) the irrigation rate (“irrigation”) in ml/min and/or the negative pressure determining the aspiration (“aspiration”) in mbar are available to the operator for this purpose.

The dependence of the device parameters on the specific situation during the surgery makes repeated adjustments of the individual settings extremely complex, and requires a high degree of experience and intuition. The available combinations of settings require a learning curve, and the settings selection in the non-sterile area requires smooth communication and cooperation with other colleagues in the operating room. In fact, in the known ultrasonic systems, there are several thousand possible combinations for the settings, of which the majority does not lead to the desired tissue effects.

### SUMMARY OF THE INVENTION

The object of the invention is therefore to provide an ultrasonic system, designed for ultrasonic surgery that makes possible a simple, safe, and efficient operation, as well as a method for its operation. In particular, an intuitive and effective operation of an ultrasonic system should also be possible with little experience with emphasis on simple operation, where even an inexperienced surgeon can direct his attention to the surgical field without constantly having to take care of the selection and setting of parameters.

The basic idea of the invention is to provide an ultrasonic system and a method for operating an ultrasonic system with which the user, i.e. the surgeon, can focus more on the operating field and has to make fewer adjustments the ultrasound system in order to achieve a certain effect. The operator must now first still categorize the tissue to be processed based on its mechanical properties. The mechanical properties can be oriented, for example, based on the modulus of elasticity of the tissue type. In the course of the surgery only the effect size resulting from the sonotrode amplitude, the irrigation rate, and/or the negative pressure, and corresponds to an ablation rate (or dissection rate). The parameters associated with the control element “effect size” comprised of the sonotrode amplitude, the irrigation rate, and/or the negative pressure, are automatically adapted to the effect size for the operator so that they no longer have to worry about setting the individual interrelated parameters. The operation of the ultrasonic system is thus greatly simplified, so that not only experienced surgeons, but also surgeons with little experience benefit from the invention, where errors can be avoided and a high level of patient care can be ensured.

According to the present the invention, an ultrasonic system is therefore proposed for ultrasonic surgery with an ultrasonic instrument including a sonotrode and a piezoelectric converter, an ultrasound generator driving the converter, and a device communicating with the ultrasonic instrument, for the supply of an irrigation fluid to the ultrasonic instrument at a predetermined irrigation rate and/or for effecting a predetermined negative pressure applied to the ultrasonic instrument, which negative pressure is configured for the aspiration of tissue fragmented by the ultrasonic instrument, in which a controller is provided which sets a current ablation rate between a predetermined minimum ablation rate and a predetermined maximum ablation rate as a function of a predetermined tissue type, in which the ablation rates are respectively determined by a value pair comprised on the one hand of the sonotrode amplitude, and on the other hand of the irrigation rate or negative pressure, or a value triplet comprised of sonotrode amplitude, irrigation rate, and negative pressure.

Thus an inventively designed ultrasonic system can be designed, for example, such that it is configured for only a certain tissue type, in which the value pair or value triplet to be set by the controller is dependent on this tissue type.

Preferably, however, the ultrasonic system has an input device for selecting a predetermined tissue type from a group of predetermined tissue types. The parameters of the value pair or of the value triplet assigned to the respective effect size that is set by means of the controller differ depending on the predetermined or selected tissue type.

For example, the user can distinguish between a soft and a hard tissue type, optionally with intermediate stages of medium hardness, that correspond in particular to moduli of elasticity, specifically in the range of 830+/−64 Pa, 1840+/−180 Pa, 5862+/−1101 Pa, and 12257+/−2708 Pa. The combinations of sonotrode amplitude, irrigation rate, and negative pressure, corresponding to the selected tissue type that are set together by the controller, lead to a lower or a higher ablation rate depending on the effect size set by the controller.

In particular, the regulating variable of the controller is respectively linked to the control values sonotrode amplitude, irrigation rate, and negative pressure, where the regulating variable sonotrode amplitude, irrigation rate, and negative pressure most preferably each follow a separate monotonic function.

A value pair can be formed in particular from the values of the sonotrode amplitude, and the irrigation rate, or the values of the sonotrode amplitude and the negative pressure. The value triplet is comprised of the values of the sonotrode amplitude, the irrigation rate, and the negative pressure interconnected via the controller. It is understood that further parameters can optionally be associated with the above-mentioned parameters; however, said parameters are essential to the execution of the invention.

A particularly safe design of the ultrasonic system for the user (and the patient) is achieved by a switch additionally being provided that limits the regulating variable of the controller to a predetermined ablation rate, in which the predetermined ablation rate is lower than the predetermined maximum ablation rate. This embodiment ensures with a simple switch that the ablation rate falls within a predetermined safety range that cannot be exceeded by the controller—at least in the case of another methodology used by the user. It is specifically intended that the settings of this “safety level” meet the clinically required minimum ablation rate of 15 mg/s for the different tissue types.

Likewise, according to the invention a method is proposed for the operation of an ultrasound system including an ultrasonic instrument comprising a sonotrode and a piezoelectric converter, an ultrasonic generator driving the converter, and a device communicating with the ultrasonic instrument that is configured for the supply of an irrigation fluid to the ultrasonic instrument at a predetermined irrigation rate and/or for effecting a predetermined negative pressure applied to the ultrasonic instrument, which negative pressure is configured for the aspiration of tissue fragmented by the ultrasonic instrument; the ultrasound system is for ultrasonic surgery by generating a predetermined sonotrode amplitude, the supply of an irrigation fluid to the ultrasonic instrument at a predetermined irrigation rate and/or effecting a predetermined negative pressure applied to the ultrasonic instrument, which negative pressure is configured for the aspiration of tissue fragmented by the ultrasonic instrument to cause a predetermined ablation rate, where the sonotrode amplitude and the irrigation rate and/or the negative pressure are connected to each other by means of a single actuator in dependence on a predetermined tissue type.

The predetermined tissue type is determined in particular by the modulus of elasticity of the tissue, wherein the predetermined tissue type can in particular be specified by the device manufacturer or selected by the user.

As previously described for the system, it is possible to select between a soft and a hard tissue type, optionally with intermediate stages of medium hardness, that correspond in particular to moduli of elasticity of 830+/−64 Pa, 1840+/−180 Pa, 5862+/−1101 Pa, and 12257+/−2708 Pa. Depending on the effect size set by the actuator, the corresponding combinations based on the selected tissue type of the sonotrode amplitude, the irrigation rate, and/or the negative pressure that are set together by means of the single actuator lead to a lower or a higher ablation rate.

The operation of the ultrasonic instrument is effected in particular by setting the actuator to a value pair or value triplet, comprised of the sonotrode amplitude, the irrigation rate, and/or negative pressure that effects a predetermined ablation rate which falls between a predetermined minimum ablation rate and a predetermined maximum ablation rate.

The sonotrode amplitude, the irrigation rate, and/or the negative pressure are specifically each monotonically linked to the actuator, where the sonotrode amplitude, the irrigation rate, and/or the negative pressure in particular each follow a separate monotonic function via the control range of the actuator.

### BRIEF DESCRIPTION OF THE DRAWINGS

In the following, the invention is explained in more detail with reference to the particularly preferably designed exemplary embodiments shown in the accompanying drawings.

FIG. 1 shows an operating unit of a first particularly preferably designed ultrasonic system according to the invention that is designed for surgical procedures on bones;

FIG. 2 shows an operating unit of a second particularly preferably designed ultrasonic system according to the invention that is provided for endoscopic neurosurgical procedures; and

FIG. 3 shows an operating unit of a third particularly preferably designed ultrasonic system according to the invention that is designed for open neurosurgical procedures.

### DETAILED DESCRIPTION OF THE PREFERRED EMBODIMENTS

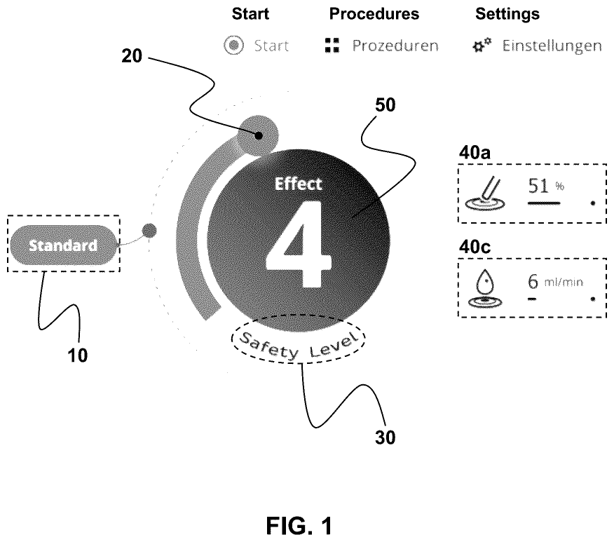

FIG. 1 shows an operating unit of a particularly preferably designed ultrasonic system according to the invention that is designed for surgical procedures on bones. In particular, FIG. 1 shows an operating unit 100, designed as a touchscreen, of a particularly preferably designed ultrasonic system according to the invention.

The operating unit 100 is specifically designed for an ultrasonic system for ultrasonic surgery on bones with an ultrasonic instrument including a sonotrode, an ultrasonic generator driving the instrument, and an irrigation device communicating with the ultrasonic instrument. The ultrasound-assisted bone instrument is an instrument developed for the dissection of bones with which the vertebrae are dissected in the context of decompressions, stabilization or fusion operations. The bones are not ground and removed by the system (as with the bone drill) but are rather cut through and can be removed in pieces. Problem osteophytes are therefore not aspirated, but rather removed in whole, remain vital, and can be reinserted into the patient. Due to the finer, but also slower working method, the bone instrument is characterized by low temperature increases. The instrument is rinsed for cooling. Depending on the localization, indication, and distance to nerves, the operator adjusts the incision speed associated with the dissection. Here, the incision speed or the pressing force required for the incision is dependent on the vibration amplitude. As the thermal load also increases with increasing amplitude, the irrigation rate is also adjusted depending on the risk assessment by the operator.

The system shown in FIG. 1 is therefore designed for a specific tissue type namely bones, which is determined in particular by a predetermined elasticity modulus. The operating unit 100 indicates the predetermined tissue type in the example shown by the display 10 with “Standard” which in the normal case cannot by changed by the operator.

The operator only has available the effect size 50, which corresponds to the ablation or dissection rate, whose amount is in turn monotonically linked to the sonotrode amplitude 40a and the irrigation rate 40c. By means of the controller 20 the operator thus only controls the level of the effect, where the sonotrode amplitude 40a and the irrigation rate 40c are controlled automatically depending on the predetermined tissue type. Here, using the switch 30 indicated with “Safety Level” the operator may limit the maximum effect size or ablation rate to a level that lies below the maximum ablation rate provided for the predetermined tissue type.

The operating concept proposed herewith thus combines the parameters sonotrode amplitude and irrigation rate depending on the predetermined tissue type and depending on the desired incision speed while optimizing the thermal load on the surrounding tissue.

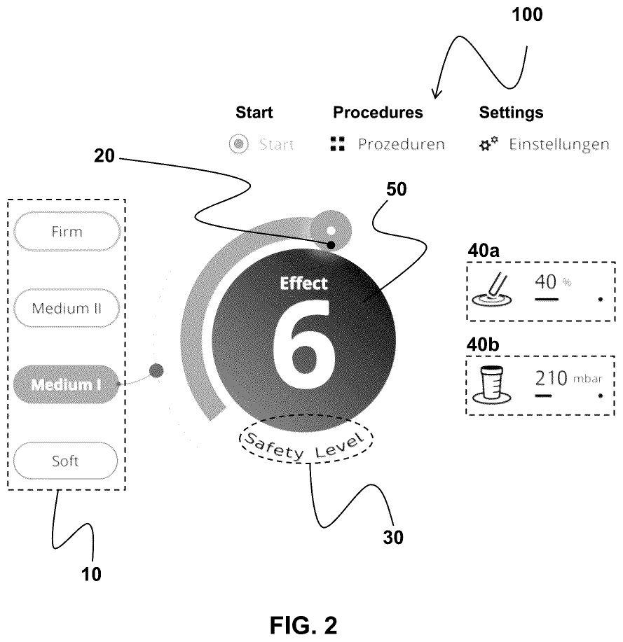

FIG. 2 shows an operating unit of a particularly preferably designed ultrasonic system according to the invention provided for endoscopic neurosurgical procedures. As above, FIG. 2 also shows an operating unit 100, designed as a touchscreen, of a particularly preferably designed ultrasonic system according to the invention.

In particular, tumors, and cysts in the fluid-filled ventricular system are ablated by means of this system. The desired ablation speed here—as in open neurosurgery—depends on the risk assessment and the desired ablation speed, which in turn depend on the consistency of the tissue. The control of the irrigation and aspiration is of particular significance, since a negative pressure in the skull can lead to a ventricular collapse. In principle, any fluid aspired should be replaced directly by fluid infusion. The fluid infusion therefore does not take place via an active pump mechanism, but rather via gravitational irrigation that is connected to the endoscope. The irrigation is therefore not controlled by the instrument or the generator, whereby only the parameters sonotrode amplitude 40a and aspiration pressure 40b remain for the new operating concept. These two parameters are combined with each other by the controller 20 depending on the tissue consistency to be selected by the operator on the input device 10, and the desired effect size 50.

Also in this case, by means of the switch 30 indicated with “Safety Level” the operator may limit the maximum effect size or ablation rate to a value that lies below the maximum ablation rate provided for the predetermined tissue type.

In any case, in the present case, after selecting the tissue type to be processed by the surgeon, the surgeon can concentrate on the operating field and need only adjust the effect size 50, by means of the controller 20 in order to cause a change of the parameters sonotrode amplitude 40a and negative pressure 40b that are monotonically linked to the controller considering exclusively advantageous combinations.

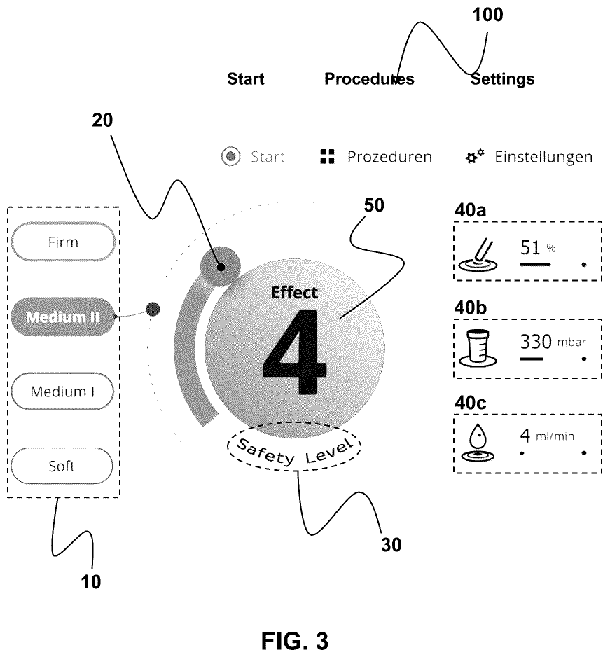

FIG. 3 finally shows the operating unit of a particularly preferably designed ultrasonic system according to the invention that is intended for use in open neurosurgery. In particular, FIG. 3 shows an operating unit 100, designed as a touchscreen of an inventively designed ultrasonic system.

The operating unit 100 is designed specifically for an ultrasonic system for neurosurgery having an ultrasonic instrument including a sonotrode, an ultrasonic generator driving the instrument, and an irrigation/aspiration device communicating with the ultrasonic instrument. The operating unit 100 includes an input device 10 for the selection of a tissue type determined by the modulus of elasticity to be processed with the ultrasonic instrument, which in the example shown is determined as “‘Soft,” “Medium I,” “Medium II,” and “Firm.” The designations “Soft,” “Medium I,” “Medium II,” and “Firm” correspond, for example, to moduli of elasticity of 830+/−64 Pa, 1840+/−180 Pa, 5862+/−1101 Pa, and 12257+/−2708 Pa.

If the operator has determined the tissue type to be processed by means of the ultrasonic system, the operator selects the effect size shown by the display 50 by means of the controller 20. A low effect size corresponds to a low ablation rate for the selected tissue type, and a high effect size to a high ablation rate in relation to the selected tissue type.

By means of the switch 30 designated with “Safety Level” the operator may limit the effect size or ablation rate to a value that lies below the maximum ablation rate intended for the tissue type.

In any case, the parameters sonotrode amplitude, irrigation rate, and negative pressure are displayed together with the controller 20 depending on the tissue type selected by means of the input device 10, are shown in the displays 40a, 40b, 40c, so that a correlation between the abstract effect size and the parameters determining the ablation rate directly results for the operator.

According to a particularly preferred design of the invention, it can additionally be provided that the operator can switch off the control system by means of the controller 20 and make changes to the sonotrode amplitude, the irrigation rate, and the negative pressure directly and independently of one another.

1. An ultrasonic system for ultrasonic surgery comprising:
an ultrasonic instrument including a sonotrode and a piezoelectric converter,
an ultrasonic generator driving the converter, and
a device, communicating with the ultrasonic instrument, for a supply of an irrigation fluid to the ultrasonic instrument at a predetermined irrigation rate and/or for effecting a predetermined negative pressure applied to the ultrasonic instrument, which negative pressure is configured for aspiration of tissue fragmented by the ultrasonic instrument, wherein depending on a predetermined tissue type, a controller setting a current ablation rate between a predetermined minimum ablation rate and a predetermined maximum ablation rate is provided, wherein the ablation rates are respectively determined by a value pair including sonotrode amplitude, and irrigation rate or negative pressure, or a value triplet comprised of sonotrode amplitude, irrigation rate, and negative pressure.

2. The ultrasonic system according to claim 1, further comprising an input device for selection of a predetermined tissue type from a group of predetermined tissue types.

3. The ultrasonic system according to claim 1, wherein the predetermined tissue type is determined by its modulus of elasticity.

4. The ultrasonic system according to claim 1, wherein a regulating variable of the controller is respectively monotonically linked to control variables including sonotrode amplitude, irrigation rate and/or negative pressure.

5. The ultrasonic system according to claim 4, wherein the control variables sonotrode amplitude, irrigation rate and negative pressure each follow a separate monotonic function.

6. The ultrasonic system according to claim 4, further comprising a switch limiting the regulating variable of the controller to a predetermined ablation rate, wherein the predetermined ablation rate is lower than the predetermined maximum ablation rate.

7. A method for operating an ultrasonic system for ultrasonic surgery including an ultrasonic instrument having a sonotrode and a piezoelectric converter, an ultrasonic generator driving the converter, and a device communicating with the ultrasonic instrument, which device is arranged to supply an irrigation fluid to the ultrasonic instrument at a predetermined irrigation rate and/or to effect a predetermined negative pressure applied to the ultrasonic instrument, which negative pressure is configured for aspiration of tissue fragmented by the ultrasonic instrument, the method comprising:
generating a predetermined sonotrode amplitude,
supplying an irrigation fluid to the ultrasonic instrument at a predetermined irrigation rate, and/or
effecting a predetermined negative pressure applied to the ultrasonic instrument, which negative pressure is configured for the aspirating tissue fragmented by the ultrasonic instrument and for effecting a predetermined ablation rate, wherein the sonotrode amplitude and the irrigation rate and/or negative pressure are interlinked by a single actuator depending on a predetermined tissue type.

8. The method according to claim 7, wherein the predetermined tissue type is determined by its modulus of elasticity.

9. The method according to claim 7, wherein the predetermined tissue type is selected by a user.

10. The method according to claim 7, further comprising setting a regulating variable to a value pair or value triplet, comprised of a sonotrode amplitude and irrigation rate, and/or negative pressure that effects a predetermined ablation rate that falls between a predetermined minimum ablation rate and a predetermined maximum ablation rate.

11. The method according to claim 7, wherein the sonotrode amplitude and the irrigation rate and/or negative pressure are each monotonically linked to the single actuator.

12. The method according to claim 7, wherein the sonotrode amplitude, the irrigation rate and/or negative pressure each follow a separate monotonic function via a regulating range of the actuator.
